(19) United States
(12) Patent Application Publication
(10) Pub. No.: US 2024/0395583 A1
(43) Pub. Date: Nov. 28, 2024

(54) METHOD FOR GENERATING REFERENCE STATUS DATA FOR MONITORING STATUS OF CHAMBER, METHOD FOR MONITORING STATUS OF CHAMBER, AND APPARATUS FOR MONITORING STATUS OF CHAMBER

(71) Applicant: AETHER, INC., Daejeon (KR)

(72) Inventors: Jaehyun KIM, Daejeon (KR); Sang Hun SEO, Daejeon (KR); Jeong Beom LEE, Daejeon (KR); Sang Won LEE, Daejeon (KR); Seunghun SEO, Hwaseong-si (KR); Myung Gon SONG, Seoul (KR)

(21) Appl. No.: 18/796,875

(22) Filed: Aug. 7, 2024

Related U.S. Application Data
(63) Continuation of application No. PCT/KR2023/006061, filed on May 3, 2023.

(30) Foreign Application Priority Data
May 3, 2022 (KR) 10-2022-0054974

(51) Int. Cl. H01L 21/67; G01N 22/02

(52) U.S. Cl. CPC H01L 21/67242; G01N 22/02; G01N 33/0081

(57) ABSTRACT

Provided is a method of monitoring a status of a chamber, the method including: acquiring a reference status data set that reflects a status of a chamber; transmitting radio waves in a specific frequency range to inside the chamber to be monitored and receiving radio waves reflected by the internal space of the chamber; generating current status data of the chamber using the received radio waves; generating monitoring information on a current status of the chamber using the reference status data set and the current status data; and providing the monitoring information.

### CROSS REFERENCE TO RELATED APPLICATIONS

This is a bypass continuation of International PCT Application No. PCT/KR2023/006061, filed on May 3, 2023, which claims priority to Republic of Korea Patent Application No. 10-2022-0054974, filed on May 3, 2022, which are incorporated by reference herein in their entirety.

### BACKGROUND OF THE INVENTION

### Field of the Invention

The present disclosure relates to a method of generating reference status data for monitoring a status of a chamber, a method of monitoring a status of a chamber, and a device for monitoring a status of a chamber.

### Description of the Related Art

In semiconductor or display manufacturing and inspection processes, it is important to check whether the statuses of equipment for the processes are within verified normal ranges and whether process conditions are properly set. For example, the status of a process in progress can be partially checked by detecting a specific physical quantity of plasma, but it is difficult to personally check a status, for example, whether equipment is properly assembled before a process starts, or whether the status of equipment is normal during a process. In the end, there is no choice but to check a process result by conducting a test run, which incurs additional cost and time. In addition, even when the process result is wrong, it is difficult to find the cause of the problem.

The present disclosure is directed to providing monitoring a status of a chamber.

The present disclosure is directed to providing monitoring a status of a chamber not only when a process is in progress, but also when a process is not in progress.

The present disclosure is directed to providing a means for comparing statuses of a plurality of chambers.

Technical problems to be solved by the present disclosure are not limited to the aforementioned technical problems, and other technical problems which are not mentioned will be clearly understood by those skilled in the art from the present disclosure.

### SUMMARY OF THE INVENTION

According to one embodiment, a method to generate a reference status data to monitor a status of a chamber, the method comprising: preparing a chamber in a first status, the chamber comprising a plurality of parts, wherein an geometry of internal space of the chamber is defined by the plurality of parts, wherein the first status is defined by a combination of geometrical status of each of the plurality of parts; transmitting radio waves in a specific frequency range to an internal space of the chamber in the first status and receiving radio waves reflected by the internal space of the chamber; and generating a first reference status data of the chamber in the first status using radio waves received by an antenna.

The method further comprises preparing a chamber in a second status in which the geometrical status of at least a portion of the plurality of parts is changed from the chamber in the first status; and generating a second reference status data by transmitting and receiving radio waves to the chamber in the second status.

The transmitting and receiving of the radio waves comprise transmitting radio waves from an outside of the chamber to the internal space of the chamber through a viewport formed in the chamber and receiving radio waves reflected by the internal space of the chamber.

According to one embodiment, a method to generate a reference status data to monitor a status of a chamber, the method comprising: preparing a chamber in a first status comprising a plurality of parts, wherein a geometrical and an electrical status of internal space of the chamber are defined by the plurality of parts, wherein the first status is defined by a combination of geometrical and electrical status of each of the plurality of parts; transmitting radio waves in a specific frequency range to an internal space of the chamber in the first status and receiving radio waves reflected by the internal space of the chamber; and generating a first reference status data of the chamber in the first status using radio waves received by an antenna.

According to one embodiment, a method to monitor a status of a chamber, the method comprising: acquiring a reference status data set that reflects a status of a chamber; transmitting radio waves in a specific frequency range to an internal space of the chamber to be monitored and receiving radio waves reflected by the internal space of the chamber; generating a current status data of the chamber using the received radio waves; generating a monitoring information on a current status of the chamber using the reference status data set and the current status data; and providing the monitoring information, wherein the reference status data set includes a plurality of reference status data reflecting a plurality of status of the chamber, wherein generating the monitoring information comprises, calculating a similarity between each of the plurality of reference status data and the current status data; and generating the monitoring information based on the similarity.

The reference status data set includes a first reference status data reflecting a first status of the chamber and a second reference status data reflecting a second status of the chamber, wherein generating the monitoring information comprises, calculating a first similarity between the first reference status data and the current status data; calculating a second similarity between the second reference status data and the current status data; and generating the monitoring information based on the first similarity and the second similarity.

The monitoring information includes information indicating that the chamber is in a status in which at least the first status and the second status are overlapped when the first similarity is greater than or equal to a first standard value and the second similarity is greater than or equal to a second standard value.

The reference status data set includes a plurality of reference status data reflecting a plurality of status of the chamber, wherein generating the monitoring information comprises, calculating a similarity between each of the plurality of reference status data and the current status data; and generating the monitoring information based on the similarity.

The reference status data set includes a plurality of reference status data reflecting a plurality of status of the chamber, wherein the method further comprises setting the current status data as new reference status data when all similarities between each of the plurality of reference status data and the current status data are less than a standard value.

The method further comprises setting the current status data as new reference status data when obtaining a user input to set the current status of the chamber as a reference status.

The specific frequency range includes a first frequency section and a second frequency section, wherein generating the monitoring information comprises: calculating a first similarity between data corresponding to the first frequency section of at least a portion of the plurality of reference status data of the reference status data set and data corresponding to the first frequency section among the current status data; calculating a second similarity between data corresponding to the second frequency section of at least a portion of the plurality of reference status data the reference status data set and data corresponding to the second frequency section among the current status data; and generating the monitoring information based on the first similarity and the second similarity.

The providing the monitoring information comprises outputting an alarm in consideration of a similarity between the reference status data set and the current status data.

The transmitting and receiving the radio waves comprises transmitting radio waves from an outside of the chamber to the internal space of the chamber through a viewport formed in the chamber and receiving radio waves reflected by the internal space of the chamber.

The reference status data set includes a first process reference status data for a first process and a second process reference status data for a second process, wherein generating the monitoring information comprises: acquiring a process information; and generating the monitoring information using one of the first process reference status data and the second process reference status data and the current status data based on the process information.

According to one embodiment, non-transitory computer readable medium storing instructions, wherein the instructions, when executed by one or more processors of the device, cause the device to perform a method may be provided.

According to one embodiment, a device that monitors a status of a chamber, the device comprising: an antenna for transmitting radio waves in a frequency range of 300 MHz or more and 30 GHz or less into inside of a chamber and receiving radio waves reflected by the internal space of the chamber; a bracket for fixing the antenna to an outside of the chamber; a signal processing unit for applying an electrical signal to the antenna and acquiring the electrical signal from the antenna; a communication unit for communicating with the outside; and a control unit for generating a monitoring information on the status of the chamber.

The bracket is designed to fix the position of the antenna to the outside of the viewport formed in the chamber.

The positional relationship between the bracket and the antenna is designed such that one end of the antenna adjacent to the viewport has a predetermined distance from one side of the viewport adjacent to the antenna.

The position of the antenna is fixed by the bracket to correspond to a viewport formed in the chamber or a separate port for the antenna and the position of the antenna is fixed by the bracket such that one end of the antenna adjacent to the viewport or a separate port for the antenna has a predetermined distance from one side of the viewport or the separate port adjacent to the antenna.

The device further comprises an electromagnetic wave shield disposed on the outside the chamber to surround the antenna.

The control unit generates a status data related to radio waves transmitted from the antenna and radio waves received by the antenna, generates the monitoring information using the status data.

The control unit generates a status data related to ratio between an input voltage applied to the antenna and an output voltage output from the antenna, generates the monitoring information using the status data.

However, the solving means of the problems of the present disclosure are not limited to the above-described solving means and solving means which have not been mentioned may be clearly understood from the disclosure by those skilled in the art.

According to the embodiments of the present disclosure, radio waves are transmitted to inside the chamber and radio waves reflected by the internal space of the chamber are received and analyzed, thereby monitoring a status of the chamber with less cost and time.

The effect of the present disclosure is not limited to the above-mentioned effect, other effects which are not mentioned will be clearly understood by those skilled in the art from the present disclosure.

### BRIEF DESCRIPTION OF THE DRAWINGS

FIG. 1 is a block diagram illustrating a monitoring device according to an embodiment;

FIGS. 2 and 3 are schematic diagrams illustrating a monitoring device installed at a chamber, according to an embodiment;

FIG. 4 is a graph illustrating S11 parameter, an example of status data;

FIGS. 5 and 6 are flowcharts illustrating a method of generating reference status data, according to an embodiment;

FIG. 7 is a diagram illustrating a reference status data set including reference status sub data sets, according to an embodiment;

FIGS. 8 and 9 are flowcharts illustrating a method of monitoring a chamber, according to an embodiment;

FIG. 10 is a diagram illustrating displaying monitoring information, according to an embodiment;

FIG. 11 is a diagram illustrating analysis for each frequency section of status data, according to an embodiment;

FIG. 12 is a block diagram illustrating a monitoring system according to an embodiment;

FIG. 13 is a schematic diagram illustrating a monitoring system installed at process equipment, according to an embodiment;

FIGS. 14 to 17F are diagrams illustrating statuses of a chamber used in experimental examples; and

FIGS. 18A to 21 are diagrams illustrating experimental results in the experimental examples.

### DETAILED DESCRIPTION OF THE INVENTION

Embodiments described in the present disclosure are for clearly describing the idea of the present disclosure to those skilled in the art to which the present disclosure belongs, so the present disclosure is not limited to the embodiments described in the present disclosure and the scope of the present disclosure should be construed as including modifications or variations that are within the idea of the present disclosure.

As the terms used in the present disclosure, general terms currently widely used are used considering functions in the present disclosure. However, the terms may vary according to the intentions of those skilled in the art, precedents, or the emergence of new technology. However, unlike this, when a particular term is used defined as having an optional meaning, the meaning of the term will be described. Thus, the terms used in the present disclosure should be construed based on the actual meanings of the terms and details throughout the present disclosure rather than simply the names of the terms.

The drawings accompanying the present disclosure are for easily describing the present disclosure, and the shapes shown in the drawings may be exaggerated to help the understanding of the present disclosure, so the present disclosure is not limited by the drawings.

In the present disclosure, if it is decided that a detailed description of known configuration or function related to the present disclosure makes the subject matter of the present disclosure unclear, the detailed description is omitted. In addition, throughout the present disclosure, the terms first, second, and so on are used only to distinguish from one element to another, unless otherwise noted.

According to the present disclosure, a monitoring device may be provided. The monitoring device transmits radio waves to inside a chamber of process equipment, such as semiconductor process equipment and display process equipment, and receives radio waves reflected by the internal space of the chamber, thereby monitoring a geometrical status of the internal space of the chamber. More specifically, the chamber includes a plurality of parts, for example, a lower electrode on which a substrate, such as a wafer, is placed, an upper electrode facing the lower electrode, pins for supporting a substrate, and a baffle. The geometrical status of the chamber may be defined by a combination of geometrical statuses, such as positions or shapes, of the respective parts. Herein, even if the same radio waves are transmitted to inside the chamber, the received radio waves may vary depending on the geometrical status of the chamber. For example, in the case in which the geometrical status of a portion of the parts is changed, for example, the position or shape of the portion of the parts is changed, even if the same radio waves are transmitted to inside the chamber, the radio waves received after change may be different from the radio waves received before change. That is, how radio waves are reflected by the internal space of the chamber may vary depending on the geometrical status of the chamber. In other words, the received reflected waves may reflect the geometrical status of the chamber.

Accordingly, the monitoring device is capable of monitoring the chamber in which a process is not in progress (as well as the chamber in which a process is in progress). For example, the monitoring device may monitor the geometrical status of the internal space of the chamber to monitor the chamber in which a process is not in progress, such as determining an assembly status of process equipment when the process equipment is manufactured, or determining a result of preventive maintenance (PM) of process equipment. As another example, the monitoring device monitors the geometrical status of the internal space of the chamber to monitor the chamber in which a process is in progress, such as proceeding a process while checking each process step proceeds normally, or predicting the timing of preventive maintenance.

The device and the method for monitoring a status of a chamber disclosed herein monitor whether equipment is properly assembled before a process starts, or whether the status of equipment is normal between processes and during a process, as described above. That is, the objects to be monitored by the device and the method disclosed herein may include the geometrical shape of each of the parts inside the chamber, a level of wear-out of each of the parts, the relative positional relationship between each of the parts, unnecessary deposition of by-products generated by the process of the target substrate through the chamber on the inner wall of the chamber and the surface of each part inside the chamber, etc.

On the other hand, the objects to be monitored by the device and the method disclosed herein may not include material supplied into the chamber to be monitored and state of energy (for example, active species, etching gas, inert gas flowing into the chamber to perform the necessary process, RF power or their plasma, etc.).

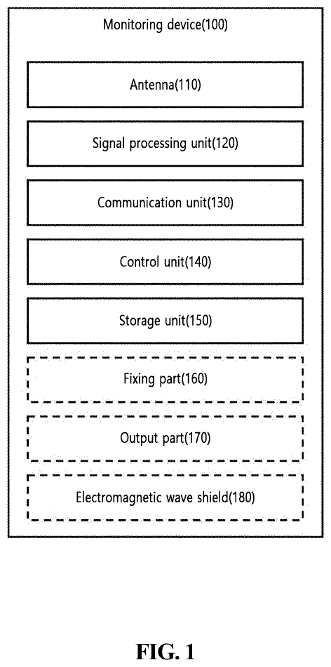

FIG. 1 is a block diagram illustrating a monitoring device according to an embodiment. Referring to FIG. 1, a monitoring device 100 may include an antenna 110, a signal processing unit 120, a communication unit 130, a control unit 140, and a storage unit 150.

The monitoring device 100 may transmit and receive radio waves through the antenna 110. The antenna 110 may receive an electrical signal to transmit radio waves. The antenna 110 may receive radio waves and convert the radio waves into an electrical signal. The monitoring device 100 may include one antenna 110. Alternatively, the monitoring device 100 may include two or more antennas 110. In this case, one or some of the two or more antennas 110 may be for transmitting radio waves and the others may be for receiving radio waves. Alternatively, each of the two or more antennas 110 may be for transmitting and receiving radio waves at different locations.

The monitoring device 100 may apply an electrical signal to the antenna 110 and obtain an electrical signal from the antenna 110 through the signal processing unit 120. The signal processing unit 120 may apply an electrical signal in a specific frequency range to the antenna 110. The signal processing unit 120 may obtain an electrical signal in a specific frequency range from the antenna 110.

The monitoring device 100 may generate status data through the signal processing unit 120. More details on this will be described later.

The monitoring device 100 may perform communication with the outside through the communication unit 130. For example, the communication unit 130 may transmit status data and monitoring information to the outside.

The communication unit 130 may perform wired or wireless communication. The communication unit 130 may be, for example, a wired/wireless local area network (LAN) module, a WAN module, an Ethernet module, a Bluetooth module, a Zigbee module, a universal serial bus (USB) module, an IEEE 1394 module, a Wi-Fi module, an Ether-CAT module, a device net module, or a combination thereof, but is not limited thereto.

The monitoring device 100 may generate monitoring information through the control unit 140. The control unit 140 may generate monitoring information based on status data. More details on this will be described later.

The control unit 140 may be realized as a computer or a similar device according to hardware, software, or a combination thereof. Hardware-wise, the control unit 140 may be one or a plurality of processors. Alternatively, the control unit 140 may be provided as processors that are physically spaced apart from each other and cooperate through communication. The control unit 140 may be, for example, a central processing unit (CPU), a graphics processing unit (GPU), a digital signal processor (DSP), a state machine, a field programmable gate array (FPGA), an application specific integrated circuit (ASIC), a radio-frequency integrated circuit (RFIC), or a combination thereof, but is not limited thereto. Software-wise, the control unit 140 may be provided in the form of a program that drives the control unit 140 that is hardware.

The monitoring device 100 may store various types of data and programs in the storage unit 150. For example, the storage unit 150 may store status data generated by the signal processing unit 120. As another example, the storage unit 150 may store monitoring information generated by the control unit 140.

The storage unit 150 may be, for example, a non-volatile semiconductor memory, a hard disk, a flash memory, a solid-state drive (SSD), a random-access memory (RAM), a read-only memory (ROM), an electrically erasable programmable read-only memory (EEPROM), other types of tangible non-volatile recording media, or a combination thereof, but is not limited thereto.

The monitoring device 100 may further include a fixing part 160. The fixing part 160 may fix the antenna 110 at a location near the chamber. For example, the fixing part 160 may fix the antenna 110 such that the antenna 110 and the chamber are spaced a predetermined distance apart. The predetermined distance may be, for example, 1 mm, 3 mm, 5 mm, 7 mm, or 1 cm, but is not limited thereto. As another example, the fixing part 160 may fix the antenna 110 such that the antenna 110 and the chamber are in contact with each other. The fixing part 160 may be, for example, a bracket, but is not limited thereto.

The monitoring device 100 may further include an output part 170. For example, the output part 170 may be a display. The monitoring device 100 may display monitoring information through the display. As another example, the output part 170 may be a speaker. The monitoring device 100 may output an alarm through the speaker.

The monitoring device 100 may further include an electromagnetic wave shield 180. The electromagnetic wave shield 180 prevents the antenna 110 from being influenced by external electromagnetic waves, or reduces the effect. For example, the electromagnetic wave shield 180 may be disposed on the outside the chamber to surround the antenna 110. The electromagnetic wave shield 180 may be provided with various materials capable of shielding electromagnetic waves.

The monitoring device 100 of an integrated type may be provided. For example, the monitoring device 100 may be provided in the form in which the antenna 110, the signal processing unit 120, the communication unit 130, the control unit 140, the storage unit 150, and the fixing part 160 are integrated.

Alternatively, the monitoring device 100 of a separated type may be provided. For example, the monitoring device 100 may be provided in the form in which the antenna 110 and the other elements are separated.

Not all the elements shown in FIG. 1 are essential elements of the monitoring device, and at least one or some of the elements of the monitoring device shown in FIG. 1 may be omitted. In addition, the monitoring device may further include elements not shown in FIG. 1.

The monitoring device may be realized using a network analyzer, such as a vector network analyzer (VNA).

For example, the signal processing unit, the communication unit, the control unit, and the storage unit may be realized using the network analyzer.

As another example, the signal processing unit may be realized using the network analyzer. In this case, in addition to the network analyzer, the monitoring device may include an additional device for realizing the communication unit, the control unit, and the storage unit.

The monitoring device may be installed in the chamber.

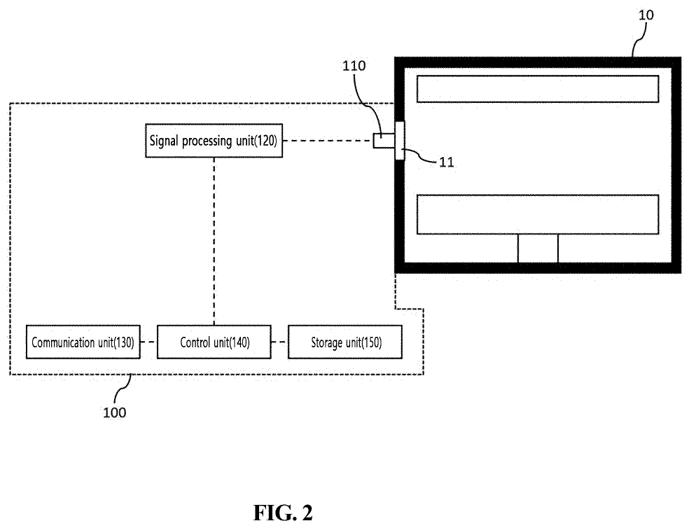

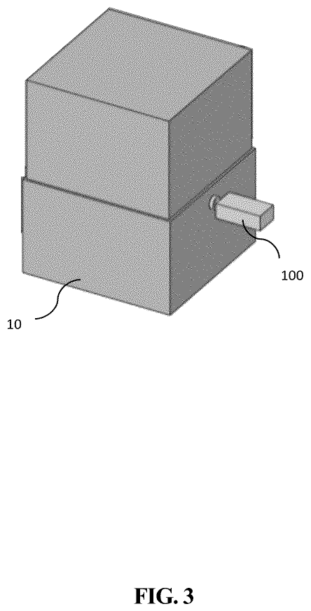

FIGS. 2 and 3 are schematic diagrams illustrating a monitoring device installed at a chamber, according to an embodiment.

Referring to FIG. 2, an antenna may be located on outside of the chamber 10. For example, the antenna may be located at the outside of a viewport 11 formed in the chamber 10.

The antenna may be installed on the outside of the chamber 10 by a fixing part. For example, the antenna may be installed on the outside of the chamber 10 by the fixing part such that the antenna and the chamber 10 are spaced a predetermined distance apart. As another example, the antenna may be installed on the outside of the chamber 10 by the fixing part such that the antenna and the chamber 10 are in contact with each other.

Although not shown, the antenna may be located inside the chamber. For example, the antenna may be located at the inside of the viewport formed in the chamber. The antenna may be installed inside the chamber by the fixing part. However, when the antenna is located inside the chamber, the antenna may contaminate the chamber or the antenna may be contaminated as a process progresses, which may be disadvantageous compared to locating the antenna on the outside of the chamber.

Referring to FIG. 3, in the case of the monitoring device 100 of the integrated type, the monitoring device 100 may be used in the form of being mounted to the chamber 10.

In the case of the monitoring device of the separated type, the monitoring device may be used in the form in which only one or some elements of the monitoring device are mounted to the chamber and the other elements are connected to the elements mounted to the chamber via wires. For example, the monitoring device may be used in the form in which the antenna is mounted to the chamber and the other elements are connected to the antenna via wires. The connecting wires may be coaxial cables, for example.

It has been described with reference to FIGS. 2 and 3 that the antenna is located corresponding to the viewport, but in addition to the viewport, the antenna may be located corresponding to an area, such as a ceramic, of the chamber through which radio waves penetrate. For example, in addition to the existing viewport, an additional port for mounting the antenna is formed in the chamber and the antenna may be located corresponding to the additional port.

In addition, it has been described with reference to FIGS. 2 and 3 that the monitoring device includes one antenna, but the monitoring device may include two or more antennas as described above. In this case, one or some of the two or more antennas may be mounted to an area of the chamber and the others may be mounted to another area of the chamber.

Hereinafter, a method of monitoring a status of a chamber will be described in more detail.

The monitoring device may generate status data.

The status data may be related to radio waves (hereinafter, referred to as “incident waves”) incident from the antenna to inside the chamber, and radio waves (hereinafter, referred to as “reflected waves”) reflected by the internal space of the chamber and received by the antenna. The status data may be related to a voltage applied to the antenna and a voltage output from the antenna.

The status data may correspond to a specific frequency range. The status data may be generated for a specific frequency range.

The status data may be defined using the incident waves and the reflected waves. For example, the status data may be defined using the ratio between the incident waves and the reflected waves, for example, the ratio of the reflected waves to the incident waves. The status data may be, for example, S-parameters, H-parameters, Y-parameters, Z-parameters, or parameters calculated therefrom, but is not limited thereto.

In some embodiments, the status data may be S-parameters.

In some other embodiments, the status data may be H-parameters.

In some other embodiments, the status data may be Y-parameters.

In some other embodiments, the status data may be Z-parameters.

In some other embodiments, the status data may be parameters derived from at least one selected from the group of S-parameters, H-parameters, Y-parameters, and Z-parameters.

In addition, in some other embodiments, the status data may be a combination of at least two parameters selected from the group of S-parameters, H-parameters, Y-parameters, and Z-parameters.

In addition, in some other embodiments, the status data may be parameters derived from a combination of at least two parameters selected from the group of S-parameters, H-parameters, Y-parameters, and Z-parameters.

That is, the status data may be i) S-parameters, ii) H-parameters, iii) Y-parameters, iv) Z-parameters, v) a combination of at least two parameters selected from the group of S-parameters, H-parameters, Y-parameters, and Z-parameters, vi) parameters derived from S-parameters, H-parameters, Y-parameters, and Z-parameters, or vii) parameters derived from a combination of at least two parameters selected from the group of S-parameters, H-parameters, Y-parameters, and Z-parameters.

For example, the status data may be represented as the following n×2 matrix.
(the first frequency) (the size of reflected waves/size of incident waves)
(the second frequency) (the size of reflected waves/size of incident waves)
. . . .
(the n-th frequency) (the size of reflected waves/size of incident waves)

As another example, the status data may be represented as the following n×3 matrix.
(the first frequency) (the size of incident waves) (the size of reflected waves)
(the second frequency) (the size of incident waves) (the size of reflected waves)
. . . .
(the n-th frequency) (the size of incident waves) (the size of reflected waves)

Herein, n denotes the number of frequencies at which the status data is measured. The first frequency is the lower limit of the frequency at which the status data is measured. The n-th frequency is the upper limit of the frequency at which the status data is measured. The first frequency to the n-th frequency are a frequency range in which the status data is measured.

The status data may include parameter values for a specific frequency range. In the above example, (the size of reflected waves/size of incident waves), (the size of incident waves), and (the size of reflected waves) may be parameter values. The number of parameter values included in the status data may be, for example, at least 500, 1000, 1500, or 2000, but is not limited thereto. Regarding the status data, a plurality of pieces of status data belonging to a specific frequency range may include a plurality of peaks belonging to the specific frequency range. The number of peaks included in the status data may be, for example, at least 5, 10, 20, 50, or 100, but is not limited thereto.

The status data may correspond to a specific time point. The status data may be generated at a specific time point. For example, the specific time point may be a time point at which a process is not in progress, for example, a time point at which assembly of process equipment is completed when the equipment is manufactured, or a time point at which preventive maintenance of process equipment is completed. As another example, the specific time point may be a time point at which a process is in progress, for example, a time point at a specific process step.

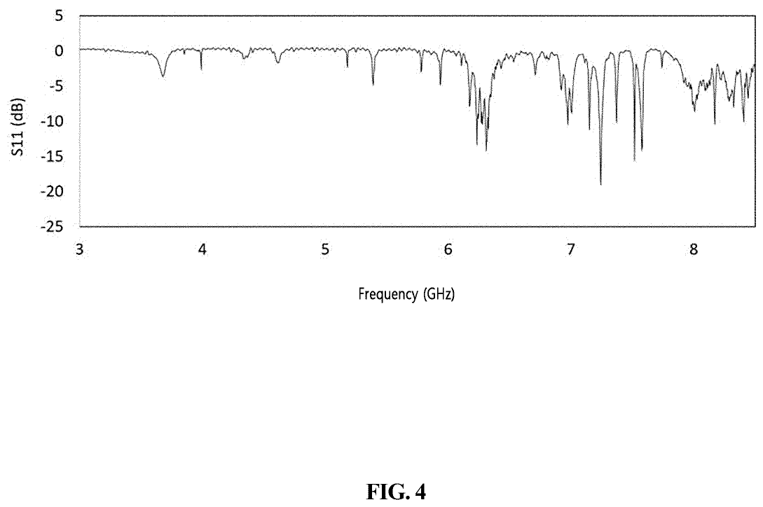

FIG. 4 is a graph illustrating S11 parameter, an example of status data. FIG. 4 shows the S11 parameter in dB for the frequency range of 3 GHz to 8.5 GHZ. In FIG. 4, it is found that there are several tens of peaks, but the number of peaks may vary depending on the criterion for counting peaks.

The status data may reflect a geometrical status of the chamber. For example, when a geometrical status of a part, such as the position or shape of the part included inside the chamber, is changed, the status data after change may be different from the status data before change. As another example, the status data when a portion of the parts is broken may be different from the status data when there is no breakage. As still another example, the status data when there is a foreign matter inside the chamber may be different from the status data when there is no foreign matter.

The status data may also reflect an electrical status of the chamber. For example, since conductivity and dielectric permittivity are depending on the material properties of the chamber or parts, the status data may reflect the electrical status of the chamber. As another example, when a polymer film is formed on the walls inside the chamber as a process progresses, the permittivity on the walls changes, so the status data may reflect the electrical status of the chamber.

According to an embodiment, the monitoring device may transmit radio waves for each frequency in a specific frequency range to inside the chamber and may receive radio waves reflected by the internal space of the chamber to generate the status data. For example, the monitoring device may transmit radio waves of a first frequency to inside the chamber and receive radio waves reflected by the internal space of the chamber to generate a first parameter value, and may transmit radio waves of a second frequency to inside the chamber and receive radio waves reflected by the internal space of the chamber to generate a second parameter value. By performing this for all the frequencies, the monitoring device may generate the status data corresponding to the specific frequency range.

To monitor the chamber, the chamber may exist in a status that is a standard for chamber monitoring, and the chamber in the status that is the standard and the chamber in the current status may be compared to monitor the chamber in the current status. In this way, the status of the chamber that is the standard for chamber monitoring may be referred to as a reference status. For example, the reference status may be a golden chamber status. As another example, the reference status may be a status of the chamber that a user is interested in. Examples of the chamber that a user is interested in may include a chamber in a good assembly status, a chamber with preventive maintenance completed, a chamber with a good process result, a chamber in a bad status, a chamber in a status in need of preventive maintenance, and a chamber in an accident status, but are not limited thereto.

The monitoring device may generate status data (hereinafter, referred to as “reference status data”) of the chamber in the reference status.

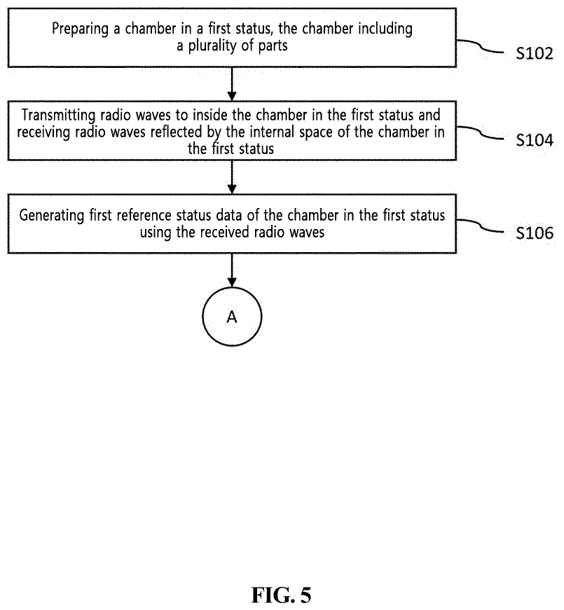

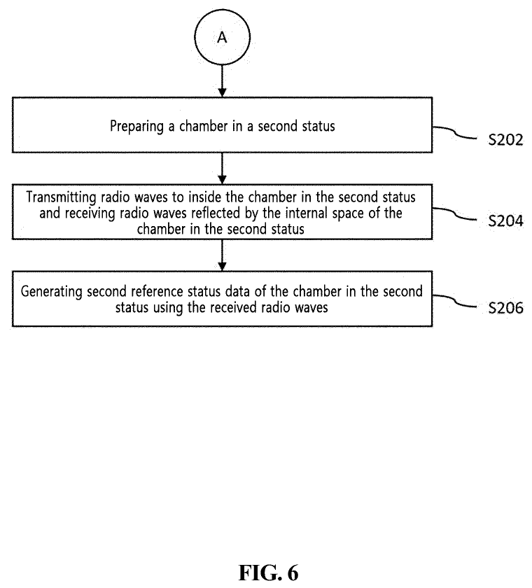

FIGS. 5 and 6 are flowcharts illustrating a method of generating reference status data, according to an embodiment.

Referring to FIG. 5, the method of generating reference status data may include: preparing a chamber in a first status, the chamber including a plurality of parts, in step S102; transmitting radio waves to inside the chamber in the first status and receiving radio waves reflected by the internal space of the chamber in the first status in step S104; and generating first reference status data of the chamber in the first status using the received radio waves in step S106.

In step S102, the monitoring device may be installed at the chamber, which includes a plurality of parts, in the first status. The chamber in the first status may be the chamber in the reference status described above.

In step S104, the monitoring device may transmit radio waves in a specific frequency range to inside the chamber in the first status and may receive radio waves reflected by the internal space of the chamber in the first status.

The specific frequency range may be determined considering the characteristics of objects to be monitored with a device and a method for monitoring a status of a chamber disclosed herein.

For example, as described above, the device and the method for monitoring a status of a chamber disclosed herein monitor whether equipment (chamber) is properly assembled before a process starts, or whether there is no abnormality on the inner wall of the equipment or the surface of various parts placed inside the equipment due to the process between processes and after completion of the process, etc. That is, the objects to be monitored by the device and the method disclosed herein may include the geometrical shape of each of the parts inside the chamber, a level of wear-out of each of the parts, the relative positional relationship between each of the parts, unnecessary deposition of by-products generated by the process of the target substrate through the chamber on the inner wall of the chamber and the surface of each part inside the chamber, etc. On the other hand, the objects to be monitored by the device and the method disclosed herein may not include material supplied into the chamber to be monitored and state of energy (for example, active species, etching gas, inert gas flowing into the chamber to perform the necessary process, RF power or their plasma, etc).

Accordingly, the specific frequency range used in the device and the method disclosed herein may be determined as a frequency band that is advantageous for monitoring the geometrical shape each of the parts inside the chamber, the relative positional relationship between each of the parts, a level of wear-out of each of the parts or unnecessary deposition of by-products generated by the process of the target substrate through the chamber on the inner wall of the chamber and the surface of each part inside the chamber.

In the meantime, the specific frequency range may be determined as a frequency band that is not affected or relatively less affected by the material supplied, during the process of the substrate, into the chamber to be monitored and affected by state of energy in the chamber (for example, active species, etching gas, inert gas flowing into the chamber to perform the necessary process, RF power or their plasma, etc).

According to some embodiments, the specific frequency range may be determined as a frequency band with wavelengths of 1 mm to 1000 mm. That is, the specific frequency range may be from 300 MHz to 300 GHz.

According to some other embodiments, the specific frequency range may be determined as a frequency band with wavelengths of 10 mm to 500 mm. That is, the specific frequency range may be from 600 MHz to 30 GHz.

According to some other embodiments, the specific frequency range may be from 1 GHz to 20 GHz.

The specific frequency range may depend on the size of the internal space of the chamber and/or the size of the parts included in the chamber.

The lower limit of the specific frequency range may depend on the size of the internal space of the chamber. For example, the larger the size of the internal space of the chamber, the smaller the lower limit.

The upper limit of the specific frequency range may depend on the size of the parts included in the chamber. For example, the smaller the size of the parts, the larger the upper limit.

Step S104 may include transmitting radio waves to inside the chamber in the first status. For example, step S104 may include transmitting radio waves from the outside of the chamber in the first status to inside the chamber in the first status.

Step S104 may include receiving radio waves reflected by the internal space of the chamber in the first status. For example, step S104 may include receiving, by the outside of the chamber in the first status, radio waves reflected by the internal space of the chamber in the first status.

In step S106, the monitoring device may generate the first reference status data of the chamber in the first status using the received radio waves. The above-described details of generating status data may be similarly applied to the generating of the first reference status data by using the received radio waves, so a redundant description thereof will be omitted.

The method of generating reference status data may proceed as shown in FIG. 6.

Referring to FIG. 6, the method of generating reference status data may include: preparing a chamber in a second status in step S202; transmitting radio waves to inside the chamber in the second status and receiving radio waves reflected by the internal space of the chamber in the second status in step S204; and generating second reference status data of the chamber in the second status using the received radio waves in step S206.

In step S202, the monitoring device may be installed at the chamber, which includes a plurality of parts, in the second status. The chamber in the second status may be the chamber in the reference status described above.

The chamber in the second status may be the chamber in a different status than the chamber in the first status.

The status of the chamber may be defined by a combination of the geometrical statuses of the parts included in the chamber as described above. Therefore, the chamber of which the geometrical status of at least a portion of the parts in different may be in a different status.

The statuses of the chamber may vary depending on the positions of the parts. For example, the chamber of which a first part is in a first position and the chamber of which the first part is in a second position may be in different statuses.

The statuses of the chamber may vary depending on the shapes of the parts. For example, the chamber of which a first part is in a normal shape and the chamber of which the first part is in an abnormal shape (for example, the part is broken) may be in different statuses. As another example, the chamber of which a first part is in a normal shape and a second part is in an abnormal shape and the chamber of which the first part is in an abnormal shape and the second part is in a normal shape may be in different statuses.

The statuses of the chamber may vary depending on whether the parts exist. For example, the chamber in which a first part exists and the chamber in which the first part does not exist (for example, the part is removed) may be in different statuses.

The statuses of the chamber may vary depending on whether foreign matters exist. For example, the chamber in which a foreign matter exists and the chamber in which the foreign matter does not exist may be in different statuses.

The above-described factors defining the statuses of the chamber may be applied independently. For example, the case in which a first part changes the status of the chamber will be described. Assuming that the first part has five positions, two shapes (normal/abnormal), and two states (existence and inexistence), the first part causes the chamber to have at least 20 (5×2×2=20) different statuses. If a second part causes the chamber to have 20 different statuses, the first part and the second part cause the chamber to have at least 400 (20×20=400) different statuses.

In step S204, the monitoring device may transmit radio waves in a specific frequency range to inside the chamber in the second status and may receive radio waves reflected by the internal space of the chamber in the second status. The above-described details of step S104 may be similarly applied to this, so a redundant description will be omitted.

In step S206, the monitoring device may generate the second reference status data of the chamber in the second status using the received radio waves. The above-described details of generating status data may be similarly applied to the generating of the second reference status data by using the received radio waves, so a redundant description thereof will be omitted.

The generating of the reference status data of the chamber in two statuses has been described with reference to FIGS. 5 and 6, but similarly to this, reference status data of the chamber in three or more statuses may be generated. By generating a plurality of pieces of reference status data as described above, a reference status data set or reference status library may be built.

Optionally, a reference status data set may include a plurality of reference status sub data sets. For example, a plurality of reference status sub data sets may be related to different processes, respectively. A reference status sub data set may include at least one piece of reference status data.

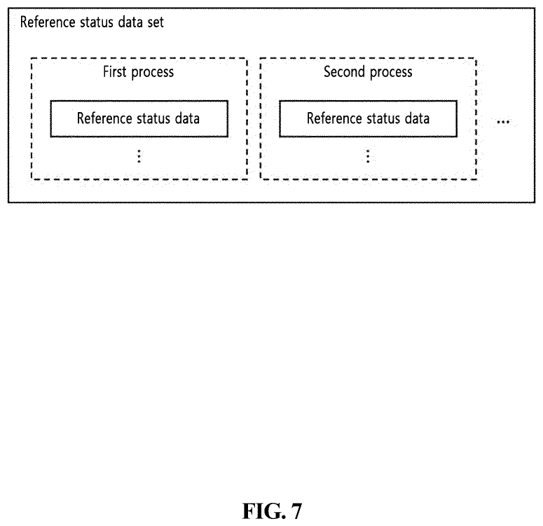

FIG. 7 is a diagram illustrating a reference status data set including reference status sub data sets, according to an embodiment. Referring to FIG. 7, the reference status data set may include a first process reference status sub data set for a first process and a second process reference status sub data set for a second process. Each of the first process reference status sub data set and the second process reference status sub data set may include at least one piece of reference status data.

In order to monitor the current status of a chamber, such as determining an assembly status of process equipment when the process equipment is manufactured, determining a result of preventive maintenance (PM) of process equipment, proceeding a process while checking whether each process step proceeds normally, or predicting the timing of preventive maintenance, the monitoring device may monitor the current status of the chamber by using reference status data and current status data of the chamber (hereinafter, referred to as “current status data”). For example, the monitoring device may compare reference status data and current status data to monitor the current status of the chamber.

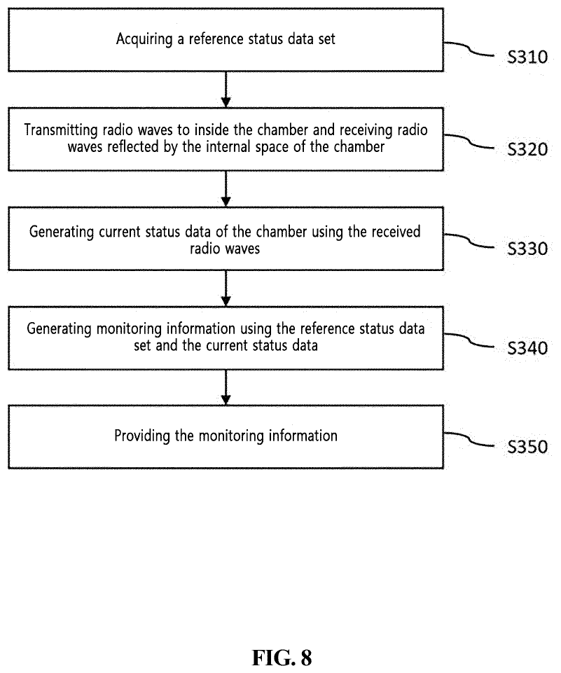

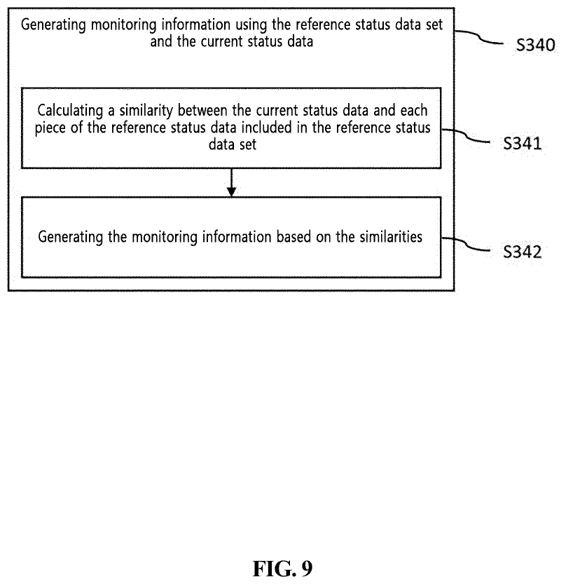

FIGS. 8 and 9 are flowcharts illustrating a method of monitoring a chamber, according to an embodiment.

Referring to FIG. 8, the method of monitoring a chamber may include: acquiring a reference status data set in step S310; transmitting radio waves to inside the chamber and receiving radio waves reflected by the internal space of the chamber in step S320; generating current status data of the chamber using the received radio waves in step S330; generating monitoring information using the reference status data set and the current status data in step S340; and providing the monitoring information in step S350.

In step S310, the monitoring device may acquire the reference status data set. The reference status data set may be generated as described above.

In step S320, the monitoring device may transmit radio waves in a specific frequency range to inside the chamber to be monitored and may receive radio waves reflected by the internal space of the chamber to be monitored. The above-described details of step S104 may be similarly applied to this, so a redundant description will be omitted.

In step S330, the monitoring device may generate the current status data of the chamber using the received radio waves. The above-described details of generating status data may be similarly applied to the generating of the current status data by using the received radio waves, so a redundant description thereof will be omitted.

In step S340, the monitoring device may generate the monitoring information using the reference status data set and the current status data.

The monitoring information may include status data.

The monitoring information may include information indicating which reference status the current status is.

The monitoring information may include information on the similarity between the reference status data and the current status data. It may be understood or determined that the higher the similarity, the more similar a current status and a reference status. Alternatively, it may be understood or determined that a current status is a reference status when the similarity exceeds a similarity standard value. In other words, the current status of the chamber may be determined by referring to the similarity.

The monitoring information may include a scatter plot of the current status data with respect to the reference status data. The scatter plot enables a user to visually determine the relationship between the reference status data and the current status data.

The monitoring information may include a history of the chamber determined as being in the reference statuses in the past. Examples of the history may include the date, time, and number of times that the reference statuses occurred, but are not limited thereto.

The monitoring information may include a description of reference statuses. The description may be about, for example, what statuses the reference statuses mean, but is not limited thereto, and may be various details related to the reference statuses.

Referring to FIG. 9, step S340 may include: calculating a similarity between the current status data and each piece of the reference status data included in the reference status data set in step S341; and generating the monitoring information based on the similarities in step S342.

In step S341, the monitoring device may calculate the similarity between the current status data and each piece of the reference status data included in the reference status data set. The monitoring device may calculate a first similarity between the current status data and first reference status data and a second similarity between the current status data and second reference status data. That is, a plurality of similarities are calculated for the current status data.

The similarities may be calculated using, for example, average, sum of squares (SOS), a graph similarity algorithm such as cosine similarity, a correlation integral technique, or a convolution technique, but no limitation thereto is imposed.

Hereinafter, some examples of calculating similarity will be described.

As an example of calculating similarity, the monitoring device may calculate similarity by using the difference between reference status data and current status data within a specific frequency range. For example, referring to Equation 1 below, the monitoring device may calculate, as similarity, the sum of squares of differences between parameter values of reference status data and parameter values of current status data within a specific frequency range.

$$Similarity = {\sum }_{n = 1}^{N} {( S 11 ( Reference status , n ) - S 11 ( Current status , n ) )}^{2} \qquad \text{<Equation 1>}$$

Herein, S11 (reference status) denotes a parameter value of reference status data, S11 (current status) denotes a parameter value of current status data, and N denotes the total number of data points.

As another example of calculating similarity, referring to Equation 2 below, the monitoring device may calculate similarity by using cosine similarity between reference status data and current status data within a specific frequency range.

$$Similarity = \frac{{S 11 ( Reference status )}^{⟶} \cdot {S 11 ( Current status )}^{⟶}}{‖ {S 11 ( Reference status )}^{⟶} ‖ ‖ {S 11 ( Current status )}^{⟶} ‖} \qquad \text{<Equation 2>}$$

Herein, S11 (reference status) represents parameter values of reference status data in a vector form, and S11 (current status) represents parameter values of current status data in a vector form.

Although the use of a plurality of pieces of reference status data has been mainly described with reference to FIGS. 8 and 9, one piece of reference status data may be used and, in this case, the above-described details may be similarly applied.

In step S350, the monitoring device may provide the monitoring information.

Step S350 may include displaying the monitoring information through a display unit.

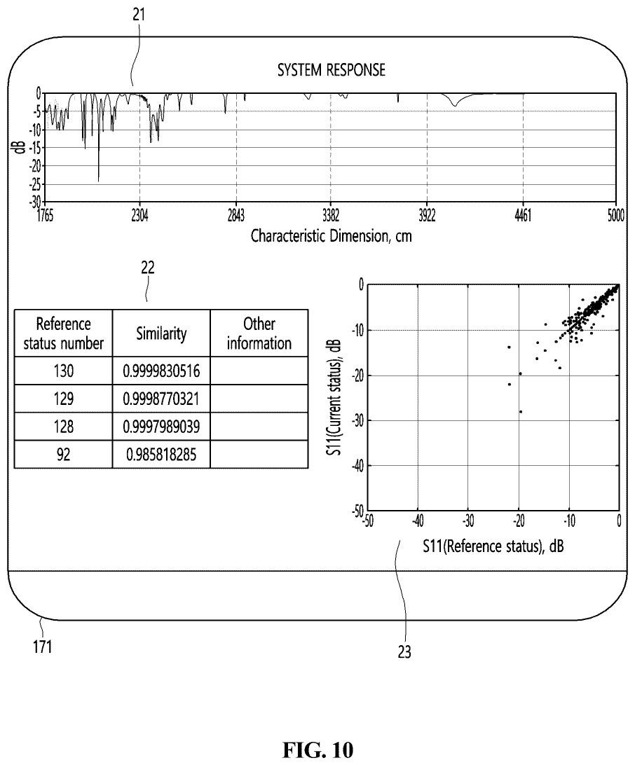

FIG. 10 is a diagram illustrating displaying monitoring information, according to an embodiment. Referring to FIG. 10, the monitoring device may display, through a display unit 171, monitoring information including status data 21, information 22 on similarity, and a scatter plot 23.

Step S350 may include transmitting the monitoring information to an external device. For example, the monitoring device may transmit the monitoring information to an external device, such as process equipment, and a fab fault detection and classification (FDC) system.

Step S350 may include outputting an alarm. Herein, outputting an alarm may mean that the monitoring device outputs an alarm directly through an output part or transmits an alarm signal to an external device.

The monitoring device may output an alarm considering the similarity between reference status data and current status data. For example, if the reference status is an abnormal status, such as an accident or dangerous situation, the monitoring device may output an alarm when the similarity exceeds a standard value. As another example, if the reference status is a normal status, such as a golden chamber status, the monitoring device may output an alarm when the similarity is less than a standard value. Herein, standard values in an abnormal status and a normal status may be the same or different from each other.

The method of monitoring the chamber may further include setting current status data as new reference status data. The monitoring device may set the current status data as new reference status data.

According to an embodiment, the monitoring device may set current status data as new reference status data on the basis of the similarity between reference status data and the current status data. For example, when all similarities between reference status data included in a reference status data set and current status data are less than a standard value, the monitoring device may set the current status data as new reference status data.

According to another embodiment, the monitoring device may acquire a user input to set a current status as a reference status and may set current status data as new reference status data. For example, when if a new type of failure occurs, a user may want to record status data in the situation to use the status data in the future. In this case, the user may input a user input to the monitoring device to set a current status as a reference status. The monitoring device that has obtained the user input may set the current status data as new reference status data.

Optionally, the monitoring device may represent a current status as a status in which two or more reference statuses are overlapped. In this case, information, included in the monitoring information, indicating which reference status the current status is may indicate that the current status is a status in which two or more reference statuses are overlapped. For example, when there are two or more reference statuses exceeding similarity standard values for a current status, the monitoring device may represent the current status as a status in which two or more reference statuses are overlapped.

Optionally, the monitoring device may analyze status data for each frequency section to calculate similarity.

In status data for a specific frequency range, the specific frequency range may be divided into a plurality of frequency sections. The monitoring device may calculate similarity for each frequency section and may monitor the chamber on the basis of similarity for each frequency section. For example, the monitoring device may monitor the chamber by directly using similarity for each frequency section. As another example, the monitoring device may calculate similarity for the entire frequency range by using similarity for each frequency section and may monitor the chamber by using the similarity for the entire frequency range.

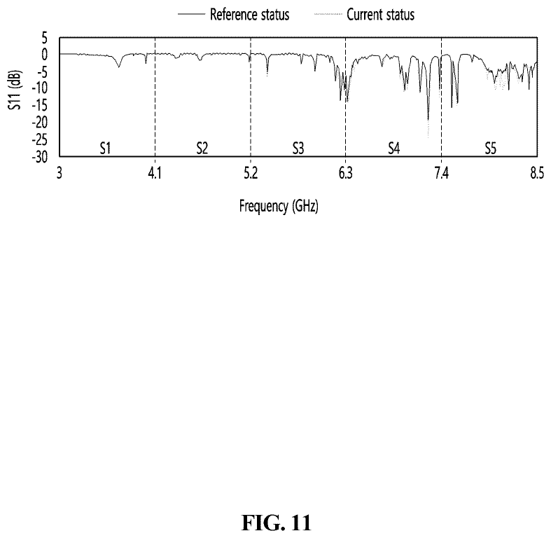

FIG. 11 is a diagram illustrating analysis for each frequency section of status data, according to an embodiment. Referring to FIG. 11, the entire frequency range from 3 GHz to 8.5 GHz may be divided into five frequency sections S1, S2, S3, S4, and S5 in 1.1 GHz increments. The monitoring device may calculate similarity for frequency section to calculate five similarities and may monitor the chamber on the basis of the five similarities.

With reference to FIG. 11, it has been described the chamber is monitored with division into five frequency sections in 1.1 GHz increments, but this is only an example, and the size or number of frequency sections are not limited thereto.

Although the monitoring of the geometrical status of the internal space of the chamber has been mainly described, the above-described method of monitoring the chamber is not limited thereto. The method of monitoring the chamber according to an embodiment may perform monitoring electrical characteristics of the internal space of the chamber. For example, the monitoring device may use the status data as described above to perform process monitoring, such as plasma monitoring or cleaning end point monitoring. As another example, the monitoring device may use the status data as described above to monitor the deposition of a material, such as polymer, on the inner walls of the chamber as a process progresses. As still another example, the monitoring device may use the status data as described above to monitor change in permittivity due to the internal space of the chamber or poor surface finish on the parts.

A monitoring system including at least one monitoring device described above may be provided. The monitoring system may be installed in process equipment including at least one chamber.

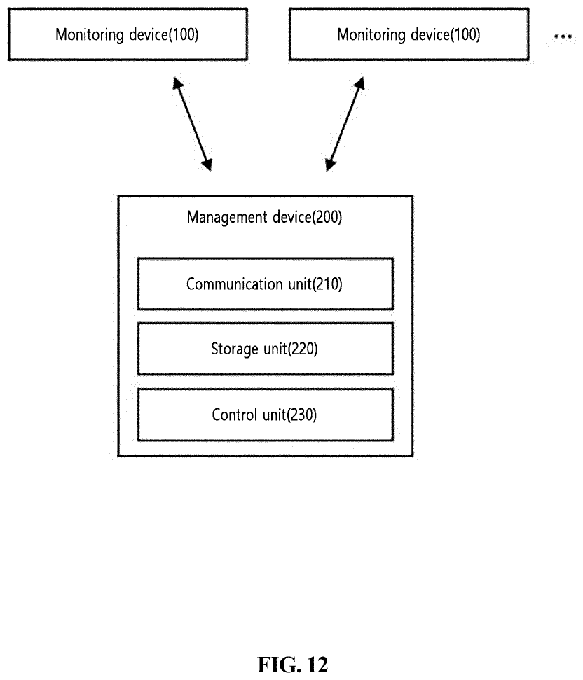

FIG. 12 is a block diagram illustrating a monitoring system according to an embodiment. Referring to FIG. 12, the monitoring system may include one or more monitoring devices 100 and a management device 200. Herein, a monitoring device 100 is the monitoring device 100 described above, so a redundant description thereof will be omitted.

The monitoring system may include the management device 200. The management device 200 may manage the monitoring devices 100.

The management device 200 may include a communication unit 210, a storage unit 220, and a control unit 230.

The management device 200 may perform communication with the outside through the communication unit 210. For example, the management device 200 may acquire monitoring information from the monitoring devices 100 through the communication unit 210. As another example, the management device 200 may transmit monitoring information to an external device, such as process equipment, a fab, through the communication unit 210. A redundant description of that similar to a communication unit 210 of a monitoring device 100 will be omitted.

The management device 200 may store various types of data and programs in the storage unit 220. For example, the storage unit 220 may store monitoring information. As another example, the storage unit 220 may store reference status data. A redundant description of that similar to a storage unit 220 of a monitoring device 100 will be omitted.

The control unit 230 may perform processing and computation of various types of information within the management device 200. The control unit 230 may control other elements constituting the management device 200. A redundant description of that similar to a control unit 230 of a monitoring device 100 will be omitted.

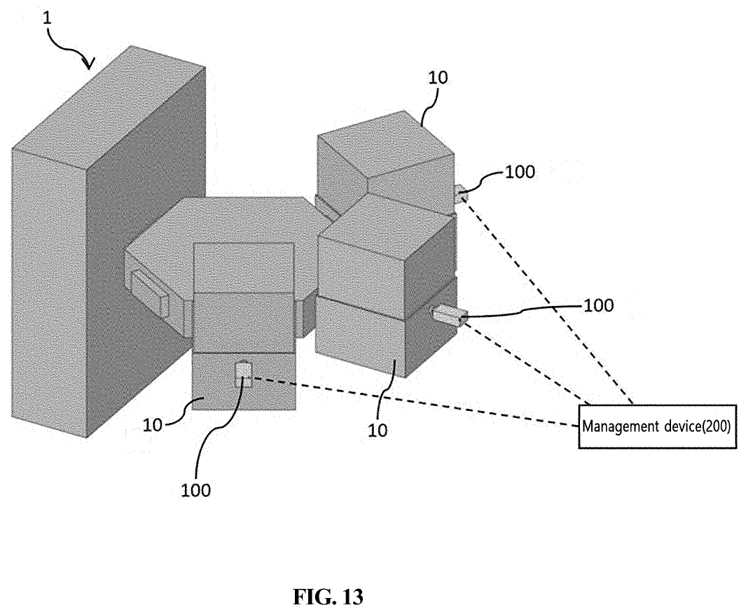

FIG. 13 is a schematic diagram illustrating a monitoring system installed at process equipment, according to an embodiment, wherein a monitoring device is of an integrated type. Referring to FIG. 13, a monitoring device 100 may be mounted to each chamber 10 of a process equipment 1. The monitoring device 100 may generate status data of the chamber 10 to which the monitoring device 100 is mounted. The monitoring device 100 may generate monitoring information based on the status data. Alternatively, the monitoring device 100 may transmit the status data to the management device 200, and the management device 200 may generate monitoring information.

A method according to an embodiment may be performed by processing logic including hardware, firmware, software, or a combination thereof. A method according to an embodiment may be performed by a processor for executing code stored in a non-transitory computer-readable medium. Examples of the non-transitory computer-readable medium include magnetic media such as hard disks, floppy disks, and magnetic tapes; optical media such as CD-ROMs, and DVD-ROMs; magneto-optical media such as floptical disks; and hardware devices, such as ROM, RAM, and flash memory, which are particularly structured to store and execute program instructions.

Although the present disclosure has been described above with reference to embodiments, the present disclosure is not limited thereto. It will be apparent to those skilled in the art to which this application belongs that various changes or modifications can be made the spirit and scope of the present disclosure, and that such changes or modifications fall within the scope of the appended patent claims.

### EXPERIMENTAL EXAMPLES

Hereinafter, experimental examples in which monitoring information, such as status data, similarity, and a scatter plot, according to a geometrical status of a chamber was generated will be described. The following experimental examples are illustrative and are not intended to limit the scope of the present disclosure in any way.

These experiments were conducted to verify whether a geometry of an internal space of a chamber could be monitored through the above-described monitoring method.

First, a chamber including three lift pins and a baffle was prepared and the following wafers were prepared: a half wafer, a prime wafer, a seasoned wafer, a wafer (PR coated wafer) coated with PR in the thickness of 700 nm, a seasoned wafer (Kapton 5% coverage seasoned wafer) with Kapton film covering 5% of area, a seasoned wafer (Kapton 15% coverage seasoned wafer) with Kapton film covering 15% of area, and a seasoned wafer (Kapton 50% coverage seasoned wafer) with Kapton film covering 50% of area. In addition, an M4 bolt was prepared.

Afterward, a monitoring device including an antenna and a VNA was prepared and installed at the chamber.

Afterward, the chamber, the wafers, and the bolt were used to prepare the chamber in 25 different statuses, and for each of the statuses, S11 parameter was measured in a frequency range of 3 GHz to 8.5 GHz to generate a reference status data set including 25 pieces of reference status data.

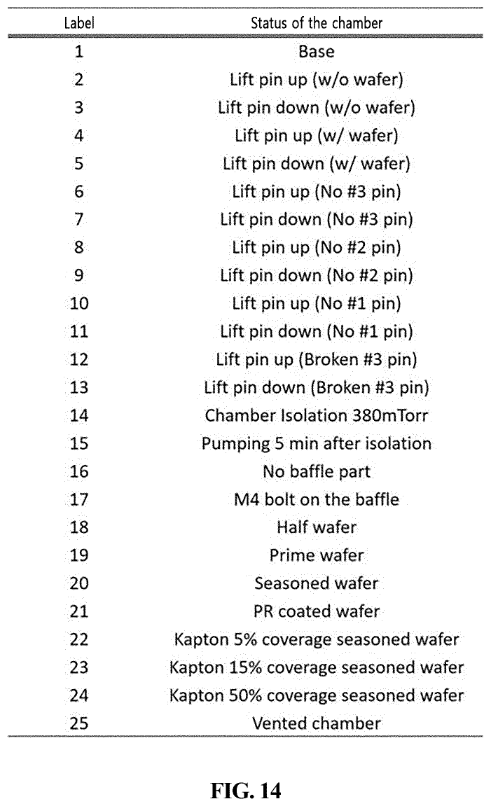

The statuses of the chamber used in the experiment are shown in FIGS. 14 to 17.

The base status refers to the status of waiting for a process while a vacuum was maintained.

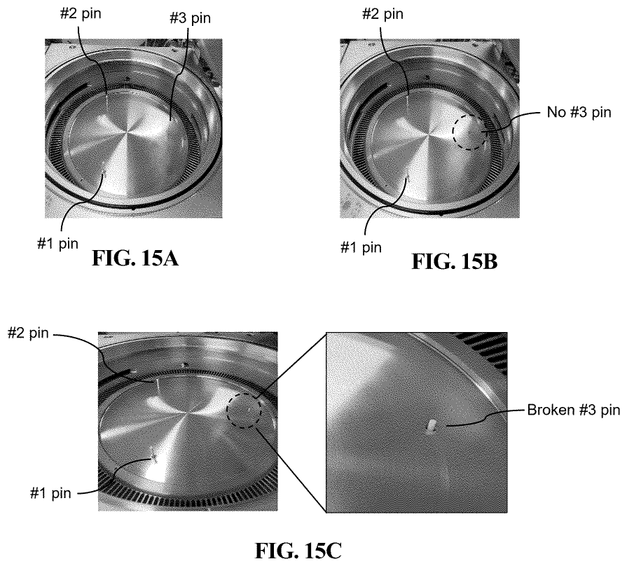

The lift pin up (w/o wafer) status refers to the status in which all the three lift pins were up without a wafer (of FIG. 15A).

The lift pin down (w/o wafer) status refers to the status in which all the three lift pins were down without a wafer.

The lift pin up (w/wafer) status refers to the status in which all the three lift pins were up and a wafer was placed on the lift pins.

The lift pin down (w/wafer) status refers to the status in which all the three lift pins were down and a wafer was placed on the lift pins.

The lift pin up (No #3 pin) status refers to the status in which lift pins were up without a wafer and there was no pin 3 (of FIG. 15B).

The lift pin down (No #3 pin) status refers to the status in which lift pins were down without a wafer and there was no pin 3.

The lift pin up (No #2 pin) status refers to the status in which lift pins were up without a wafer and there was no pin 2.

The lift pin down (No #2 pin) status refers to the status in which lift pins were down without a wafer and there was no pin 2.

The lift pin up (No #1 pin) status refers to the status in which lift pins were up without a wafer and there was no pin 1.

The lift pin down (No #1 pin) status refers to the status in which lift pins were down without a wafer and there was no pin 1.

The lift pin up (Broken #3 pin) status refers to the status in which lift pins were up without a wafer and pin 3 was broken (FIG. 15C).

The lift pin down (Broken #3 pin) status refers to the status in which lift pins were down without a wafer and pin 3 was broken.

The chamber isolation 380 mTorr status refers to the status in which after the chamber was isolated by closing a valve on a flow path to a pump in the base status, the internal pressure was increased to 380 mTorr.

The pumping 5 min after isolation status refers to the status in which 5 minutes passed after isolation in the chamber isolation 380 mTorr status was cancelled and the valve on the flow path to the pump was opened to start pumping.

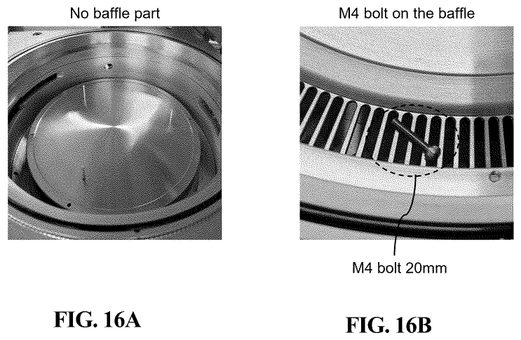

The no baffle part status refers to the status of the chamber in which there was no baffle (of FIG. 16A).

The M4 bolt on the baffle status refers to the status of the chamber in which the M4 bolt was placed on the baffle (of FIG. 16B).

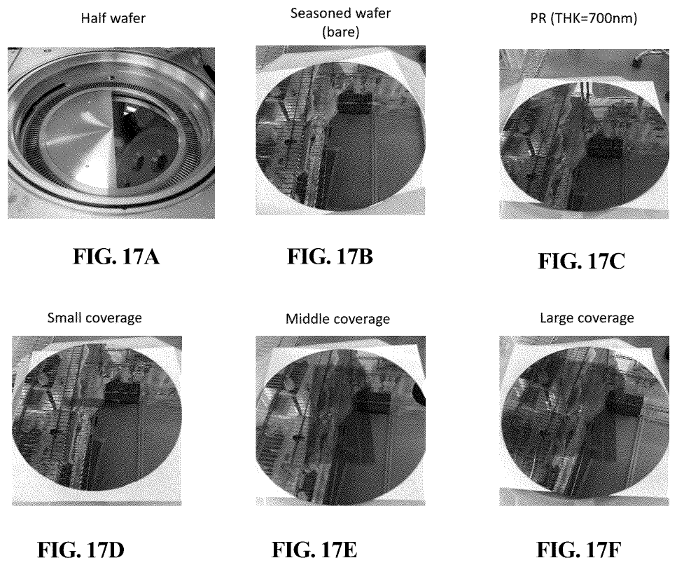

The half wafer status refers to the status in which the half wafer was placed in the chamber (of FIG. 17A).

The prime wafer status refers to the status in which the prime wafer was placed in the chamber.

The seasoned wafer status refers to the status in which the seasoned wafer was placed in the chamber (of FIG. 17B).

The PR coated wafer status refers to the status in which the PR coated wafer was placed in the chamber (of FIG. 17C).

The Kapton 5% coverage seasoned wafer status refers to the status in which the seasoned wafer with the Kapton film covering 5% of area was placed in the chamber (of FIG. 17D).

The Kapton 15% coverage seasoned wafer status refers to the status in which the seasoned wafer with the Kapton film covering 15% of area was placed in the chamber (of FIG. 17E).

The Kapton 50% coverage seasoned wafer status refers to the status in which the seasoned wafer with the Kapton film covering 50% of area was placed in the chamber (of FIG. 17F).

The vented chamber status refers to the status in which the chamber in the base status was vented and the atmospheric pressure was reached.

In the above-described statuses of the chamber, the lift pins were down unless otherwise noted.

Afterward, any status was set as a current status, and a scatter plot of current status data with respect to reference status data and similarity therebetween were generated. Herein, the similarity was calculated using the sum of squares according to Equation 1 and cosine similarity according to Equation 2.

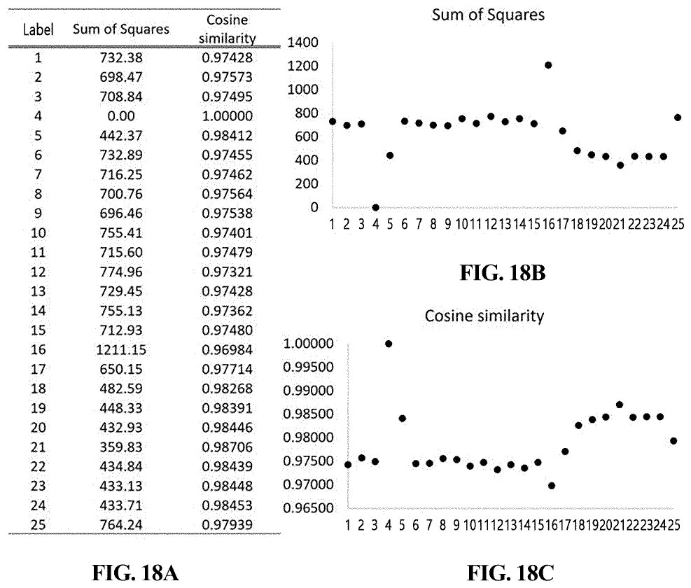

FIGS. 18A through 18C are diagrams illustrating similarity when the Lift pin up (w/wafer) status (status 4) was set as a current status. FIG. 18A shows a table of similarity, FIG. 18B shows a graph of sum-of-squares similarity, and FIG. 18C shows a graph of cosine similarity. In FIGS. 18A through 18C, it is found that status 4 was well distinguished from the other statuses.

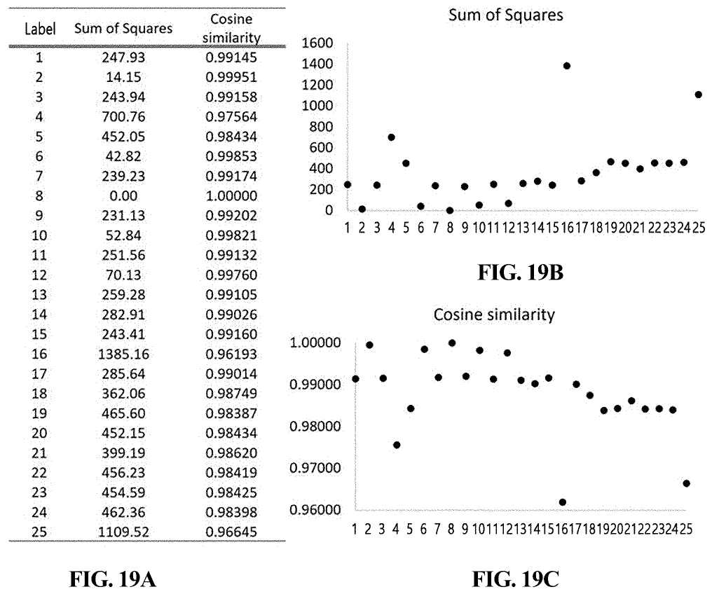

FIGS. 19A through 19C are diagrams illustrating similarity when the lift pin up (No #2 pin) status (status 8) was set as a current status. FIG. 19A shows a table of similarity, FIG. 19B shows a graph of sum-of-squares similarity, FIG. 19C shows a graph of cosine similarity. In FIGS. 19A through 19C, status 8 was similar to some statuses, but was distinguished considering the sum-of-squares similarity of 10 or greater and the cosine similarity to the fourth decimal place.

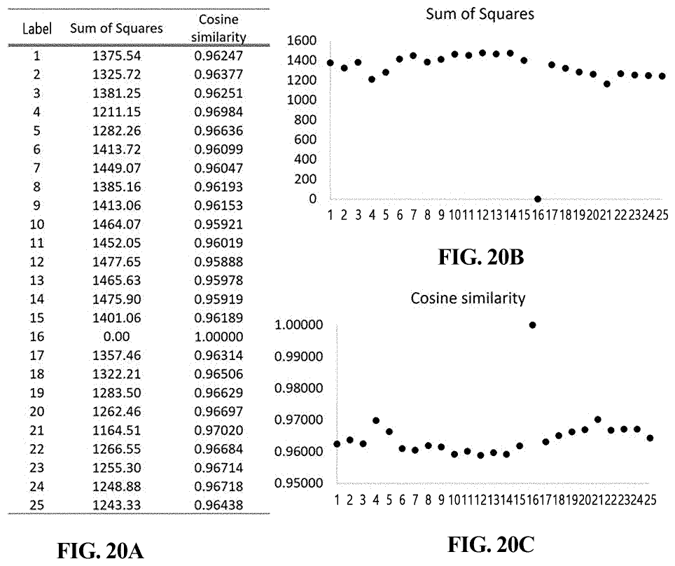

FIGS. 20A through 20C are diagrams illustrating similarity when the no baffle part status (status 16) was set as a current status. FIG. 20A shows a table of similarity, FIG. 20B shows a graph of sum-of-squares similarity, FIG. 20C shows a graph of cosine similarity. In FIGS. 20A through 20C, it is found that status 16 was better distinguished from the other statuses than in FIGS. 18A through 18C.

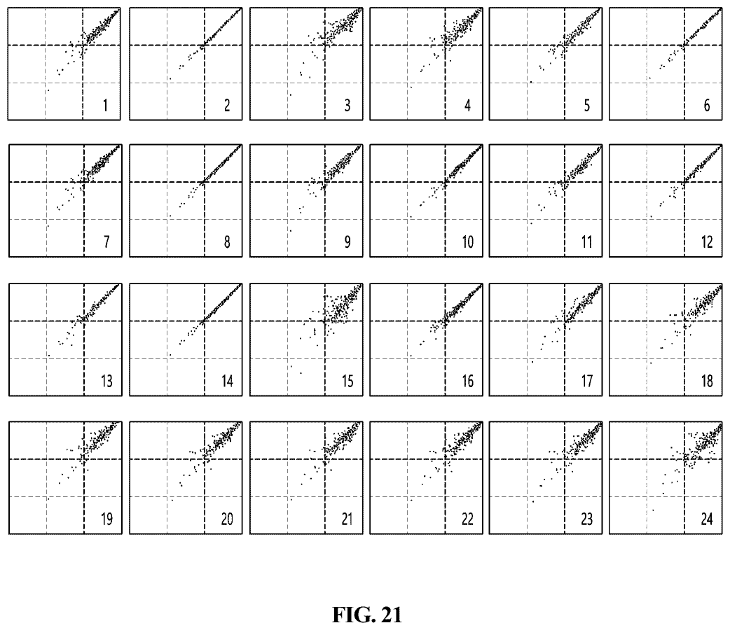

FIG. 21 is a diagram illustrating scatter plots using the base status (status 1) as a reference status with respect to the other statuses. The labels shown in the respective scatter plots are the same those shown in FIG. 14. In FIG. 21, it is visually found that the scatter plots varied from status to status.

What is claimed is:

1. A method to generate a reference status data to monitor a status of a chamber, the method comprising:
preparing the chamber in a first status, the chamber comprising a plurality of parts,
wherein a geometry of internal space of the chamber is defined by the plurality of parts,
wherein the first status is defined by a combination of geometrical status of each of the plurality of parts;
transmitting radio waves in a frequency range of 300 MHz or more and 30 GHz or less from outside or inside of the chamber in the first status to an internal space of the chamber in the first status and receiving radio waves reflected by the internal space of the chamber;
calculating one parameter selected from a group consisting of i) S-parameters, ii) H-parameters, iii) Y-parameters, iv) Z-parameters and v) one parameter derived from the S-parameters, the H-parameters, the Y-parameter and the Z-parameter within the frequency range using radio waves received by an antenna; and
generating a first reference status data of the chamber in the first status using the calculated parameter.

2. The method of claim 1,
preparing the chamber in a second status in which the geometrical status of at least a portion of the plurality of parts is changed from the chamber in the first status; and
generating a second reference status data by transmitting and receiving radio waves to the chamber in the second status.

3. The method of claim 1,
wherein the transmitting and the receiving of the radio waves comprises transmitting radio waves from an outside of the chamber to the internal space of the chamber through a viewport formed in the chamber and receiving radio waves reflected by the internal space of the chamber.

4. A method to generate a reference status data to monitor a status of a chamber, the method comprising:
preparing the chamber in a first status comprising a plurality of parts,
wherein a geometrical and an electrical status of internal space of the chamber are defined by the plurality of parts,
wherein the first status is defined by a combination of geometrical and electrical status of each of the plurality of parts;
transmitting radio waves in a frequency range of 300 MHz or more and 30 GHz or less from outside or inside the chamber in the first status to an internal space of the chamber in the first status and receiving radio waves reflected by the internal space of the chamber;
calculating one parameter selected from a group consisting of i) S-parameters, ii) H-parameters, iii) Y-parameters, iv) Z-parameters and v) one parameter derived from the S-parameters, the H-parameters, the Y-parameter and the Z-parameter within the frequency range using radio waves received by an antenna; and
generating a first reference status data of the chamber in the first status using the calculated parameter.

5. A method to monitor a status of a chamber, the method comprising:
acquiring a reference status data set that reflects the status of the chamber;
transmitting radio waves in a specific frequency range to an internal space of the chamber to be monitored and receiving radio waves reflected by an internal space of the chamber;
generating a current status data of the chamber using the received radio waves;
generating a monitoring information on a current status of the chamber using the reference status data set and the current status data; and
providing the monitoring information,
wherein the reference status data set includes a plurality of reference status data reflecting a plurality of status of the chamber,
wherein generating the monitoring information comprises,
calculating a similarity between each of the plurality of reference status data and the current status data; and
generating the monitoring information based on the similarity.

6. The method of claim 5,
wherein the reference status data set includes a first reference status data reflecting a first status of the chamber and a second reference status data reflecting a second status of the chamber,
wherein generating the monitoring information comprises,
calculating a first similarity between the first reference status data and the current status data;
calculating a second similarity between the second reference status data and the current status data; and
generating the monitoring information based on the first similarity and the second similarity.

7. The method of claim 6,
wherein the monitoring information includes information indicating that the chamber is in a status in which at least the first status and the second status are overlapped when the first similarity is greater than or equal to a first standard value and the second similarity is greater than or equal to a second standard value.

8. The method of claim 5,
wherein the reference status data set includes the plurality of reference status data reflecting the plurality of status of the chamber,
wherein the method further comprises setting the current status data as new reference status data when all similarities between each of the plurality of reference status data and the current status data are less than a standard value.

9. The method of claim 5, further comprising:
setting the current status data as new reference status data when obtaining a user input to set the current status of the chamber as a reference status.

10. The method of claim 5,
wherein the specific frequency range includes a first frequency section and a second frequency section,
wherein generating the monitoring information comprises:
calculating a first similarity between data corresponding to the first frequency section of at least a portion of the plurality of reference status data of the reference status data set and data corresponding to the first frequency section among the current status data;
calculating a second similarity between data corresponding to the second frequency section of at least a portion of the plurality of reference status data the reference status data set and data corresponding to the second frequency section among the current status data; and
generating the monitoring information based on the first similarity and the second similarity.

11. The method of claim 5,
wherein providing the monitoring information comprises outputting an alarm in consideration of a similarity between the reference status data set and the current status data.

12. The method of claim 5,
wherein the transmitting and the receiving radio waves comprises transmitting radio waves from an outside of the chamber to the internal space of the chamber through a viewport formed in the chamber and receiving radio waves reflected by the internal space of the chamber.

13. The method of claim 5,
wherein the reference status data set includes a first process reference status data for a first process and a second process reference status data for a second process,
wherein generating the monitoring information comprises:
acquiring a process information; and
generating the monitoring information using one of the first process reference status data and the second process reference status data and the current status data based on the process information.

14. A non-transitory computer readable storage medium storing instructions, wherein the instructions, when executed by one or more processors of a device, cause the device to perform a method according to claim 1.

15. A device that monitors a status of a chamber, the device comprising:
an antenna for transmitting radio waves in a frequency range of 300 MHz or more and 30 GHz or less into inside of the chamber and receiving radio waves reflected by an internal space of the chamber;
a bracket for fixing the antenna to an outside of the chamber;
a signal processing unit for applying an electrical signal to the antenna and acquiring the electrical signal from the antenna;
a communication unit for communicating with the outside; and
a control unit for generating a monitoring information on the status of the chamber,
wherein the control unit calculates one parameter selected from a group consisting of i) S-parameters, ii) H-parameters, iii) Y-parameters, iv) Z-parameters and v) one parameter derived from the S-parameters, the H-parameters, the Y-parameter and the Z-parameter within the frequency range using radio waves received by the antenna,
generates a current status data for the chamber using the calculated parameter,
generates the monitoring information based on the generated current status data and a pre-stored reference status data set.

16. The device of claim 15,
wherein the bracket is designed to fix a position of the antenna to an outside of a viewport formed in the chamber.

17. The device of claim 16,
wherein a positional relationship between the bracket and the antenna is designed such that one end of the antenna adjacent to the viewport has a predetermined distance from one side of the viewport adjacent to the antenna.

18. The device of claim 15,
wherein a position of the antenna is fixed by the bracket to correspond to a viewport formed in the chamber or a separate port for the antenna.

19. The device of claim 16,
wherein the position of the antenna is fixed by the bracket such that one end of the antenna adjacent to the viewport or a separate port for the antenna has a predetermined distance from one side of the viewport or the separate port adjacent to the antenna.

20. The device of claim 15, further comprises,
an electromagnetic wave shield disposed on the outside of the chamber to surround the antenna.

21. The device of claim 15,
wherein the control unit generates a status data related to the radio waves transmitted from the antenna and the radio waves received by the antenna, generates the monitoring information using the status data.

22. The device of claim 15,
wherein the control unit generates a status data related to ratio between an input voltage applied to the antenna and an output voltage output from the antenna, generates the monitoring information using the status data.
